(19) United States
(12) Patent Application Publication
(10) Pub. No.: US 2010/0256246 A1
(43) Pub. Date: Oct. 7, 2010

(54) SYSTEM AND METHOD FOR CONDITIONING BIOMASS-DERIVED SYNTHESIS GAS

(75) Inventors: Benjamin H. CARRYER, Denver, CO (US); Eric R. ELROD, Arvada, CO (US); Mark D. IBSEN, Highlands Ranch, CO (US); Brian K. JOHNSON, Arvada, CO (US); Harold A. WRIGHT, Longmont, CO (US)

Correspondence Address: CONLEY ROSE, P.C.;David A. Rose, P. O. BOX 3267, HOUSTON, TX, 77253-3267, US

(73) Assignee: RENTECH, INC., Los Angeles, CA (US)

(21) Appl. No.: 12/754,797

(22) Filed: Apr. 6, 2010

Related U.S. Application Data
(60) Provisional application No. 61/166,851, filed on Apr. 6, 2009.

(51) Int. Cl. C07C 1/04; C01B 3/38; B01J 19/00

(52) U.S. Cl. 518/702; 252/373; 422/189

(57) ABSTRACT

A thermal conversion process comprising: pyrolizing or gasifying a carbonaceous feedstock to produce a first synthesis gas having a first H₂:CO ratio of less than a minimum value or greater than a maximum value; providing enriched oxygen; and subjecting the first synthesis gas to partial oxidation in the presence of at least a portion of the enriched oxygen to produce a conditioned synthesis gas having a desired ratio of H₂:CO in the range of from the minimum value to the maximum value. A method of producing FT product liquids by providing a conditioned synthesis gas according to the process and producing FT product liquids by subjecting the conditioned synthesis gas to FT reaction under FT operating conditions. A system for carrying out the methods is also provided.

### CROSS-REFERENCE TO RELATED APPLICATIONS

This application claims the benefit under 35 U.S.C. §119(e) of U.S. Provisional Patent Application No. 61/166,851 filed Apr. 6, 2009, the disclosure of which is hereby incorporated herein by reference.

### STATEMENT REGARDING FEDERALLY SPONSORED RESEARCH OR DEVELOPMENT

Not applicable.

### BACKGROUND OF THE INVENTION

1. Technical Field of the Invention

The present invention relates to a method of conditioning biomass-derived synthesis gas. More specifically, the method is suitable for providing conditioned synthesis gas suitable for production of Fisher-Tropsch fuel. Still more specifically, the method comprises conditioning biomass-derived synthesis gas via thermal conversion with enriched oxygen.

2. Background of the Invention

A major drawback with biomass gasification and/or pyrolysis technology is that the hydrogen (H₂) to carbon monoxide (CO) ratio is generally too low while the content of methane and higher hydrocarbons as well as the carbon dioxide content are undesirably high for downstream processes such as Fisher-Tropsch (FT) synthesis.

Conventional methods of controlling the ratio of H₂/CO is by the use of water gas shift (WGS) reaction over catalyst to obtain the ratio needed for FT production. This method consumes CO which is a FT fuel feedstock, and produces CO₂ which is an undesired feedstock component for some FT processes. Not only does water gas shift (WGS) of biomass-derived syngas consume CO in the process while producing CO₂, which reduces FT production capacity and is an undesired component for the feedstock of certain Fischer-Tropsch processes, but the WGS also fails to reform undesired methane (and higher hydrocarbons) into H₂ and CO for use as a feedstock in a Fischer-Tropsch process. The un-reacted methane instead acts as an inert for the Fischer-Tropsch process, lowering carbon utilization and conversion to Fischer-Tropsch fuels.

A related method of H₂/CO control is provided in U.S. Patent Application No. U.S./2007/0175095. The '095 patent application discloses utilization of pure oxygen or air for use in a reforming tower to raise the temperature of a synthesis gas such that tars are thermally cracked. Other work in this field has been primarily to remove tars in biomass-derived synthesis gas. Biomass-derived synthesis gas is often intended to be used directly for the production of electricity through combustion of the synthesis gas. Thus, reduction of methane levels in biomass-derived synthesis gas has not been taught, as such reduction is not advantageous to such processes.

Accordingly, there remains a need for systems and methods of conditioning biomass-derived synthesis gas to provide conditioned synthesis gas suitable for production of Fischer-Tropsch fuel. Utilization of the synthesis gas for the production of FT liquid fuels requires conditioning of the synthesis gas to provide a desired ratio of hydrogen to carbon monoxide. Conditioning may comprise conversion of the methane (which may be considered an inert to the FT production process) and higher hydrocarbons into additional H₂ and CO.

### SUMMARY

Herein disclosed are a system and process for conditioning synthesis gas (e.g., biomass-derived synthesis gas) via thermal conversion with enriched oxygen to provide conditioned synthesis gas suitable, for example, for production of Fischer-Tropsch fuels.

Herein disclosed is a thermal conversion process comprising: providing a first synthesis gas having a first H₂:CO ratio of less than a minimum value or greater than a maximum value; providing enriched oxygen; and subjecting the first synthesis gas to partial oxidation in the presence of at least a portion of the enriched oxygen to produce a conditioned synthesis gas having a desired ratio of H₂:CO in the range of from the minimum value to the maximum value. In embodiments, the minimum value is about 0.7 and the maximum value is about 2.0. In embodiments, the minimum value is about 0.7 and the maximum value is about 1.5. In embodiments, the minimum value is about 0.75 and the maximum value is about 1.1.

In applications, the partial oxidation reaction is carried out in a reactor. In embodiments, the thermal conversion process is a non-catalytic, high temperature process. The high temperature may be a temperature in the range of from about 950° C. to about 1500° C. The high temperature may be a temperature in the range of from about 950° C. to about 1400° C.

The process may further comprise adjusting the portion of enriched oxygen based on a desired H₂:CO ratio. In embodiments, providing enriched oxygen comprises providing enriched oxygen at a flow rate in the range of from about 10 lb/h per ton of dry biomass feed to about 100 lb/h per ton of dry biomass feed. In embodiments, providing enriched oxygen comprises Vacuum Swing Adsorption (VSA). The enriched oxygen can comprise from about 50 vol % to about 100 vol % oxygen, alternatively from about 50 vol % to about 95 vol % oxygen. The enriched oxygen can further comprise nitrogen and trace gases present in air.

In applications, providing the first synthesis gas comprises pyrolizing or gasifying a carbonaceous feedstock. The method can further comprise adjusting the moisture content of the first synthesis gas by adjusting the moisture content of the carbonaceous feedstock. In embodiments, the first synthesis gas is obtained via gasification. In embodiments, the carbonaceous feedstock comprises biomass.

In applications, the conditioned synthesis gas is suitable for FT liquids production. In embodiments, the conditioned synthesis gas has a H₂:CO ratio on the range of from about 0.75 to about 1.1. In embodiments, the conditioned synthesis gas has a H₂:CO ratio on the range of from about 1.5 to about 2.0. In embodiments, the first synthesis gas has a H₂:CO ratio in the range of from 0.3 to 1.0 on a dry basis.

Also disclosed herein is a method of producing FT product liquids, the method comprising: (a) providing a conditioned synthesis gas according to the disclosed process; and (b) producing FT product liquids by subjecting the conditioned synthesis gas to FT reaction under FT operating conditions. In embodiments, the method further comprises cooling the conditioned synthesis gas via production of high pressure steam, low pressure steam, or a combination thereof. In embodiments, the tar content in the conditioned synthesis gas is less than 10% of the tar content in the first syngas. In embodiments, the method further comprises compressing the cooled conditioned synthesis gas prior to (b).

Also disclosed herein is a system for conditioning synthesis gas for production of liquid hydrocarbons via FT synthesis, the system comprising: enriched oxygen production apparatus configured to provide enriched oxygen from air; and a synthesis gas conditioning reactor fluidly coupled with the enriched oxygen production apparatus, wherein the synthesis gas conditioning reactor is configured for subjecting a first synthesis gas having a first H₂:CO ratio outside a desired range to partial oxidation in the presence of at least a portion of the enriched oxygen to produce a conditioned synthesis gas having a second H₂:CO within the desired range. In embodiments, the desired range is from about 0.75 to about 2.

The enriched oxygen production apparatus can comprise vacuum swing adsorption. In embodiments, the enriched oxygen comprises from about 50 vol % to about 100 vol % oxygen. In embodiments, the synthesis gas conditioning reactor is operable in the absence of catalyst. In embodiments, the synthesis gas conditioning reactor is operable at a temperature in the range of from about 950° C. to about 1500° C.

The system may further comprise synthesis gas production apparatus configured for the production of the first synthesis gas from a carbonaceous material. The synthesis gas production apparatus may comprise a gasifier. In embodiments, the carbonaceous material comprises biomass.

In applications, the system further comprises at least one FT reactor downstream of the synthesis gas conditioning reactor and configured for the production of FT hydrocarbons from the conditioned synthesis gas. In embodiments, the at least one FT reactor comprises FT catalyst. The FT catalyst can be iron-based.

The system can further comprise at least one heat exchange device configured for the production of high pressure steam or low pressure steam via heat transfer from the conditioned synthesis gas.

The various embodiments of the present invention overcome the various aspects of the deficiencies of the prior art and provide new and economical systems and methods for conditioning synthesis gas for use in Fischer-Tropsch processes.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more detailed description of the preferred embodiments of the present invention, reference will now be made to the accompanying drawing, in which:

FIG. 1 is a flow diagram of a system for conditioning synthesis gas according to an embodiment of this disclosure.

### NOTATION AND NOMENCLATURE

Unless noted otherwise, reference to ‘the ratio’ (for example, the ratio of hydrogen to carbon monoxide in synthesis gas) is intended to refer to mole ratios.

### DETAILED DESCRIPTION

Overview. Herein disclosed are a system and method for conditioning synthesis gas for use in a Fischer-Tropsch process. The disclosed method utilizes a non-catalytic high temperature thermal conversion process using an enriched oxygen source for the conversion of methane and higher hydrocarbons in a biomass-derived synthesis gas from a gasification and or pyrolysis process into H₂ and CO, thus providing an optimal H₂/CO ratio, suitable for use in downstream processes, for example, downstream FT synthesis. The H₂/CO ratio of biomass-derived synthesis gas is adjusted (e.g., increased) via partial oxidation of the biomass-derived synthesis gas with an enriched oxygen/nitrogen stream and reforming of methane into H₂ and CO.

Also disclosed is a system for carrying out the method, the system comprising a thermal conversion reactor (also referred to herein as a syngas conditioning reactor). The reactor is configured to convert methane and higher hydrocarbons from a biomass-derived syngas into H₂ and CO, in the absence of catalyst. The system is configured to provide a desired H₂/CO ratio, suitable for use in a downstream FT synthesis process.

The system and method utilize an enriched oxygen stream (e.g., with an oxygen content in the range of 50-100 vol %, or 50-95 vol % with the remaining comprising of nitrogen and/or other trace gases in the inlet air) to perform thermal conversion of a syngas stream (e.g., a biomass-derived synthesis gas) in the absence of a catalyst. Via the disclosed syngas conditioning system and method, the H₂/CO ratio of the feed synthesis gas may be adjusted (e.g., optimized) for use in FT processes.

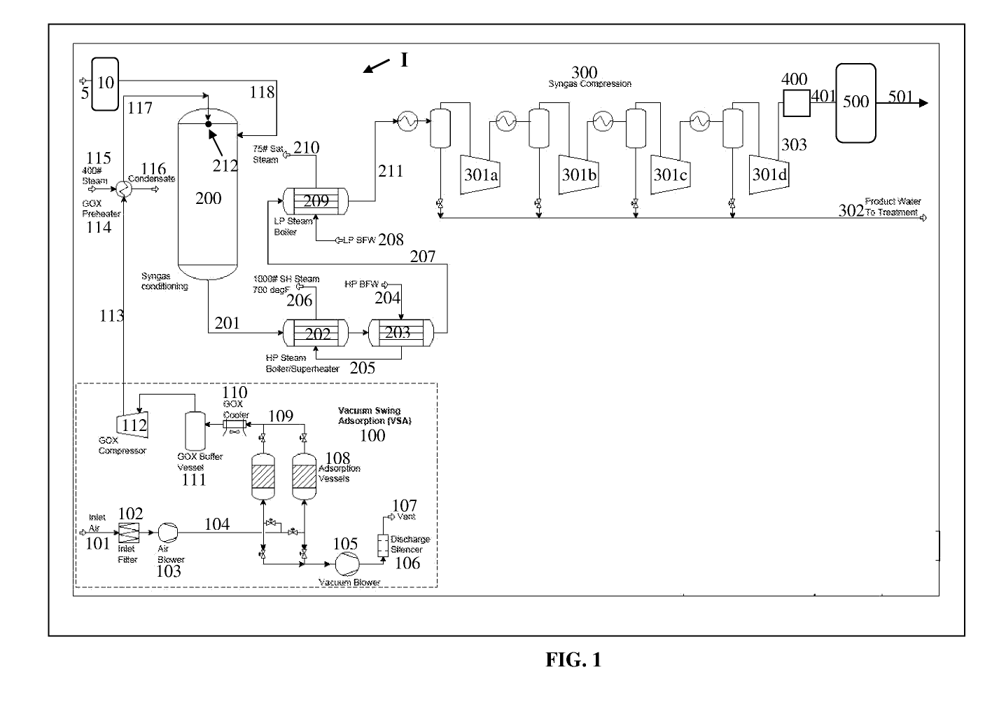

System. FIG. 1 is a flow diagram of a system I according to an embodiment of this disclosure. System I comprises enriched oxygen production apparatus 100 and synthesis gas conditioning reactor 200. Although it is envisaged that enriched oxygen production apparatus 100 may be any apparatus suitable for producing high purity oxygen from air, description will be made wherein enriched oxygen production apparatus 100 comprises vacuum swing adsorption (VSA) apparatus. System I may further comprise syngas production apparatus 10, synthesis gas compression apparatus 300, one or more additional synthesis gas cleanup units as indicated as 400, one or more Fischer-Tropsch reactors 500, or a combination thereof.

VSA apparatus 100 is configured to provide an enriched oxygen stream 113 from inlet air 101. VSA apparatus 100 may be any VSA apparatus known in the art to provide enriched oxygen from inlet air. VSA apparatus 100 comprises at least one adsorption vessel 108 (two indicated in FIG. 1). VSA apparatus 100 may further comprise inlet filter 102, air blower 103, vacuum blower 105, discharge silencer 106, vent line 107, oxygen gas or GOX cooler 110, GOX buffer vessel 111, GOX compressor 112, or some combination thereof. These unit components may be connected as indicated in FIG. 1.

Vacuum Swing Adsorption (VSA) apparatus 100 provides an enriched oxygen stream. The enriched oxygen stream may comprise from about 50% to about 100% O₂ by volume, alternatively, from about 50% to about 95% O₂ by volume. The enriched oxygen may further comprise nitrogen and trace gases present in air.

System I further comprises synthesis gas conditioning reactor 200. Synthesis gas conditioning reactor 200 is coupled with VSA apparatus 100. For example, as indicated in the embodiment of FIG. 1, outlet line 113 carrying enriched oxygen from VSA adsorption is connected with an inlet of synthesis gas conditioning reactor 200 via GOX preheater 114 and line 117. Synthesis gas conditioning reactor 200 is configured for subjecting a first synthesis gas to partial oxidation in the presence of at least a portion of the enriched oxygen produced in VSA apparatus 100 to produce a conditioned synthesis gas having a desired ratio of H₂:CO. An inlet line 118 may introduce the first synthesis gas into the syngas conditioning reactor 200. Inlet line 118 may be connected with a synthesis gas production apparatus 10, as discussed further hereinbelow. Enriched oxygen is introduced into syngas conditioning reactor 200 via enriched oxygen line 113 from VSA apparatus 100. Synthesis gas conditioning reactor 200 can comprise one or more burners 212 at which partial synthesis gas to be conditioned and enriched oxygen are intimately contacted. In embodiments, synthesis gas conditioning reactor 200 comprises a plurality of burners distributed along the top of reactor 200. In embodiments, synthesis gas conditioning reactor 200 comprises at least one burner having a diameter of at least 2 inches, at least three inches or at least four inches. In embodiments, synthesis gas conditioning reactor 200 comprises at least 2, at least 5, at least 10, at least 20, at least 50, or at least 100 burners. The burners may be positioned in any suitable arrangement within reactor 200. In embodiments, burner(s) 112 are circumferentially distributed at the top of reactor 200. In embodiments, burner(s) 112 are distributed uniformly about a cross-section of reactor 200.

The first synthesis gas may have a first H₂:CO ratio of less than a minimum value or greater than a maximum value, and syngas conditioning reactor 200 is operable to provide a conditioned synthesis gas having a desired H₂:CO ratio in the range between the minimum value and the maximum value. The first synthesis gas may be a product of pyrolizing or gasifying a carbonaceous feedstock to produce the first synthesis gas. In embodiments, the carbonaceous feedstock is biomass.

System I may further comprise synthesis gas production apparatus 10. Synthesis gas production apparatus is configured for producing the first synthesis gas from a carbonaceous feedstock introduced thereto via carbonaceous material inlet line 5. In embodiments, synthesis gas production apparatus 10 comprises a gasifier.

System I may further comprise a GOX preheater 114 configured for heating the GOX prior to introduction into synthesis gas conditioning reactor 200. Steam may be introduced into GOX preheater 114 and condensate produced via heat transfer to the enriched oxygen in line 113 removed from GOX preheater 114 via condensate line 116. Preheated enriched oxygen may be introduced into syngas conditioning reactor 200 via line 117.

System I may further comprise one or more heat transfer devices configured for removal of heat from the conditioned synthesis gas produced in synthesis gas conditioning unit 200. For example, in the embodiment of FIG. 1, boiler 203, high pressure (HP) steam boiler/superheater 202, and low pressure (LP) steam boiler 209 are configured for production of steam from boiler feed water via heat transfer with conditioned synthesis gas exiting synthesis gas conditioning reactor 200 via outlet line 201.

System I may further comprise synthesis gas compression apparatus 300. Synthesis gas compression apparatus 300 is positioned downstream of synthesis gas conditioning apparatus 200 and may be positioned downstream of one or more heat transfer devices (e.g., boiler 203, HP steam boiler/superheater 202, and/or LP steam boiler 209). Synthesis gas compression apparatus 300 comprises one or more compressor configured for compressing conditioned synthesis gas or cooled/conditioned synthesis gas. In the embodiment of FIG. 1, synthesis gas compression apparatus 300 comprises four compressors, 301a, 301b, 301c, and 301d.

System I may further comprise additional syngas cleanup units 400. Such units may be configured for removing one or more undesirable components from the conditioned synthesis gas prior to downstream FT synthesis. Additional syngas cleanup units 400 may be downstream of syngas compression apparatus 300, downstream of one or more heat removal units (e.g., boiler 203, HP steam boiler/superheater 202, and/or LP steam boiler 209), or both. Additional syngas cleanup units 400 may comprise, for example, one or more AGR units. A line 303 may be configured to introduce compressed conditioned synthesis gas into additional synthesis gas cleanup unit(s) 400.

System I may further comprise one or more FT reactor 500. FT reactor 500 is any reactor known in the art to be suitable for the production of liquid hydrocarbons from synthesis gas. In embodiments, FT reactor 500 contains therein a bed of FT catalyst. The FT catalyst may be supported or unsupported. In applications, the FT catalyst is a precipitated, supported catalyst. In applications, the FT catalyst is a precipitated, unsupported catalyst. In embodiments, the catalyst is an iron-based FT catalyst. In embodiments, the iron-based catalyst is promoted with potassium and/or copper. A line 401 may be configured to introduce conditioned synthesis gas into FT reactor 500. One or more outlet lines 501 may be coupled with FT reactor 500 for removal of FT products therefrom.

Method. Description of the method of this disclosure will now be with reference to FIG. 1. The method comprises: providing a first synthesis gas having a first H₂:CO ratio of less than a minimum value or greater than a maximum value; providing enriched oxygen; and subjecting the first synthesis gas to partial oxidation in the presence of at least a portion of the enriched oxygen to produce a conditioned synthesis gas having a desired ratio of H₂:CO in the range of from the minimum value to the maximum value.

Providing Synthesis Gas to be Conditioned. The disclosed method comprises providing a synthesis gas to be conditioned, the synthesis gas to be conditioned having a first H₂:CO ratio of less than a minimum value or greater than a maximum value. The synthesis gas to be conditioned in syngas conditioning reactor 200 may be the product of pyrolysis and/or gasification of a carbonaceous feedstock. In embodiments, the carbonaceous feedstock comprises biomass. In applications, providing syngas to be conditioned comprises introducing carbonaceous feedstock into syngas production unit(s) 10 via carbonaceous feedstock inlet line 5, and operating the syngas production unit(s) such that the feedstock is converted to synthesis gas to be conditioned. The synthesis gas to be conditioned may be obtained via gasification. The synthesis gas to be conditioned in syngas conditioning reactor 200 may have a first H₂:CO ratio of less than a minimum value or greater than a maximum value. In applications, the minimum value is about 0.7 and the maximum value is about 2.0. In embodiments, the minimum value is about 0.7 and the maximum value is about 1.5. In applications, the minimum value is about 0.75 and the maximum value is about 1.1. In embodiments, the moisture content of the synthesis gas to be conditioned is controlled by reducing the moisture content of the feed (e.g. of the biomass) introduced into the synthesis gas production unit(s). For example, the moisture content of biomass fed to a gasifier may be controlled to obtain synthesis gas, to be conditioned, having a suitable moisture content.

Providing Enriched Oxygen. The disclosed method comprises providing enriched oxygen. Enriched oxygen may be provided by any means known in the art. In embodiments, providing enriched oxygen comprises utilizing Vacuum Swing Adsorption (VSA). In embodiments, air is introduced via air inlet 101 into VSA apparatus 100. The air is introduced into one or more adsorption vessels 108. The inlet air may be filtered via passage through one or more inlet filters 102. Air blower 103 may be used to provide the inlet air to the one or more adsorption vessels 108 via line 104. Enriched oxygen exits the one or more adsorption vessels 108 via line 109. Waste gas may be sent via vacuum blower 105 and/or discharge silencer 106 to vent line 107.

Enriched oxygen exiting adsorption vessels 108 via line 109 may be cooled via passage through GOX cooler 110, stored as desired in buffer vessel 111, and/or compressed via GOX compressor 112 prior to introduction into synthesis gas conditioning unit 200.

Conditioning Synthesis Gas. The method further comprises subjecting the first synthesis gas to partial oxidation in the presence of at least a portion of the enriched oxygen to produce a conditioned synthesis gas having a desired ratio of H₂:CO in the range of from the minimum value to the maximum value. At least a portion of the enriched oxygen from VSA apparatus 100 is introduced via lines 113 and 117 into syngas conditioning unit 200. In embodiments, enriched oxygen is provided to syngas conditioning reactor 200 at a flow rate of at least 5,000 lb/h. In embodiments, enriched oxygen is provided to syngas conditioning reactor 200 at a flow rate of at least 10,000 lb/h. In embodiments, enriched oxygen is provided to syngas conditioning reactor 200 at a flow rate of at least 20,000 lb/h. In embodiments, enriched oxygen is provided to syngas conditioning reactor 200 at a flow rate in the range of from about 10,000 lb/h to about 100,000 lb/h. In embodiments, enriched oxygen is provided to syngas conditioning reactor 200 at a flow rate of at least 5 lb/h per ton of dry biomass feed. In embodiments, enriched oxygen is provided to syngas conditioning reactor 200 at a flow rate of at least 10 lb/h per ton of dry biomass feed. In embodiments, enriched oxygen is provided to syngas conditioning reactor 200 at a flow rate of at least 20 lb/h per ton of dry biomass feed. In embodiments, enriched oxygen is provided to syngas conditioning reactor 200 at a flow rate in the range of from about 10 lb/h per ton of dry biomass feed to about 100 lb/h per ton of dry biomass feed.

Synthesis gas to be conditioned is concurrently introduced into syngas conditioning unit 200 via syngas inlet line 118. The enriched oxygen stream is used for partial oxidation of hydrogen with the oxygen. The enriched oxygen may contact the synthesis gas to be conditioned at least one, at least 2, at least 5, or more burner(s) 112, as described hereinabove.

In embodiments, the hydrogen is supplied via an upstream biomass gasifier/pyrolysis reaction. In embodiments, the upstream biomass gasifier/pyrolysis reactor produces synthesis gas with a H₂/CO ratio which is less than 0.7 or greater than 1.5. Within syngas conditioning unit 200, the low H₂/CO ratio biomass-derived syngas (BDS) reacts in a partial oxidation reaction of hydrogen gas H₂ with oxygen O₂ in a reactor to produce water and heat (H₂ and O₂ are consumed in this process). In embodiments, the amount of enriched oxygen added to the syngas conditioning reactor 200 via enriched oxygen line 113 and/or 117 is based on the desired H₂/CO ratio in the conditioned synthesis gas exiting reactor 200 via line 201. The method may thus further comprise adjusting the portion (amount) of enriched oxygen based on a desired H₂:CO ratio in the conditioned syngas.

Subjecting the first synthesis gas (i.e., the synthesis gas to be conditioned) to partial oxidation may be carried out in a reactor, i.e. in embodiments, syngas conditioning reactor 200 comprises a reactor. In embodiments, the thermal conversion process performed in syngas conditioning reactor 200 is a non-catalytic, high temperature process. In applications, the high temperature is a temperature in the range of from about 950° C. to about 1500° C. or from about 950° C. to about 1100° C.

The amount of water in the synthesis gas to be conditioned (e.g., the biomass-derived synthesis gas) may be controlled, as too much water in the syngas may not allow the process to achieve the lower H₂/CO ratio (e.g., 0.75 to 1.4) range needed for certain FT processes. Controlling the water upstream and carefully controlling the addition of enriched oxygen to consume/react with some of the hydrogen in the synthesis gas produces heat and water. This will allow for the steam methane reaction to occur, consuming some methane and water to produce CO and H₂, and producing synthesis gas having a desired H₂/CO ratio. In embodiments, the synthesis gas to be conditioned (i.e., the first synthesis gas) comprises H₂O, and the method further comprises removing at least a portion of the H₂O from the first synthesis gas prior to partial oxidation. In embodiments, the moisture content of the synthesis gas to be conditioned is controlled by adjusting the moisture content of a carbonaceous feedstock from which the synthesis gas to be conditioned is derived (e.g. via gasification of the carbonaceous feedstock).

The thermal conversion process in syngas conditioning reactor 200 provides the heat and steam required for steam methane reforming of the methane and other higher hydrocarbons in the supplied BDS to form H₂ and CO to the degree that it optimizes the carbon efficiency of the biomass feedstock for production of Fisher-Tropsch liquids (e.g., providing a ratio of H₂ and CO in the range of from about 0.75 to about 2.0).

As discussed herein, the conditioned synthesis gas is suitable for FT liquids production, in embodiments of the disclosed method. In embodiments, the conditioned synthesis gas has a ratio of H₂/CO in the range of from about 0.75 to about 1.1, suitable, for example, for a downstream FT process. In embodiments, the conditioned synthesis gas may have a ratio of H₂/CO in the range of from about 1.5 to about 2.0 H₂/CO ratio, and may be suitable, for example, for use with microchannel reactors. In applications, the synthesis gas to be conditioned has an H₂/CO ratio in the range of from about 0.3 to 1 on a dry basis, and the conditioned synthesis gas has a an H₂/CO ratio in the range of from about 0.75 to about 2. For example, in applications, enriched oxygen from a VSA unit is introduced into syngas conditioning reactor 200 with a biomass-derived synthesis gas having an H₂/CO ratio in the range of from about 0.3 to about 1 on a dry basis. Adding enriched oxygen from a VSA unit and conditioning can yield a conditioned synthesis gas with a H₂/CO ratio of between 0.75 and 2. In embodiments, the product conditioned syngas has a concentration of less than about 20%, 15%, 13%, or 10% inerts (including CO₂). In embodiments, the conditioned synthesis gas comprises less than about 50%, 40%, 30%, 20%, 10% or 5 weight percent of the tar in the synthesis gas to be conditioned. In embodiments, the conditioned synthesis gas comprises less than about 10% of the tar content of the synthesis gas to be conditioned. In embodiments, conditioning provides at least 70%, 80%, 85%, 90% or 95% reduction in tar.

Cooling Conditioned Synthesis Gas. The method may further comprise cooling the conditioned synthesis gas. Cooling the conditioned syngas may be performed concomitantly with the production of high and/or low pressure steam. For example, in the embodiment of FIG. 1, conditioned syngas from syngas conditioning reactor 200 is introduced via reactor outlet line 201 into HP steam boiler/superheater 202 and further into boiler 203. HP boiler feedwater is introduced into boiler 203 via HP BFW line 204. Heat exchange within boiler 203 produces heated fluid which is introduced via line 205 into HP steam boiler/superheater 202. Heat exchange within HP steam boiler/superheater 202 produces superheated steam, which exits HP steam boiler/superheater 202 via HP steam line 206. The warm synthesis gas is introduced via line 207 into LP steam boiler 209. Boiler feed water is introduced into LP steam boiler 209 via LP BFW line 208. Low pressure steam is formed by heat transfer between the warm syngas and the LP BFW, and exits LP steam boiler 209 via LP steam line 210.

Compressing Conditioned Synthesis Gas. The method may further comprise compressing the conditioned synthesis gas. Following conditioning, the conditioned syngas, which may have been cooled as described, may be introduced into synthesis gas compression apparatus 300. The conditioned syngas is compressed via one or more compressors 301. For example, the cooled conditioned syngas exiting LP steam boiler 209 via line 211 is introduced via line 211 sequentially into four compressors 301a, 301b, 301c and 301d in the embodiment of FIG. 1. Product water may be sent to treatment and/or disposal via product water line 302.

Cleaning Up Conditioned Synthesis Gas. The method may further comprise subjecting the conditioned syngas to further cleanup. For example, one or more component may be removed from the conditioned synthesis gas. In embodiments, additional syngas cleanup is performed via one or more additional synthesis gas cleanup units 400. Unit(s) 400 may comprise, for example, AGR unit(s). Additional syngas cleanup unit is performed downstream of syngas conditioning reactor 200. Additional syngas cleanup may be performed downstream of one or more heat exchanger (e.g., boiler 202, 203, and/or 209), downstream of syngas compression apparatus 300, or downstream of both. In the embodiment of FIG. 1, compressed conditioned syngas is introduced via line 303 into additional cleanup unit(s) 400.

Producing Fischer-Tropsch Hydrocarbons. The method may further comprise producing FT hydrocarbons from the conditioned syngas. Producing FT hydrocarbons may comprise introducing the conditioned syngas into one or more FT reactor(s). In the embodiment of FIG. 1, cleaned-up synthesis gas exiting additional cleanup unit(s) 400 is introduced via line 401 into FT reactor 500. FT reactor(s) 500 is operated under FT synthesis conditions to convert the conditioned syngas into liquid hydrocarbons. FT product hydrocarbons exit FT reactor(s) 500 via one or more FT product lines 501.

Additional Features/Advantages. Use of the disclosed system and method may increase plant yield per unit feedstock. The process may be applicable in numerous biomass-derived syngas to FT fuels projects as well as in other syngas-derived chemical processes.

### EXAMPLE

### Example 1

A synthesis gas is conditioned according to the disclosed method. Parameters for the conditioning and results are presented in Tables 1 and 2 below. A syngas derived from biomass feedstock is fed to a conditioning reactor at a flow rate of 97,780 lb/h. The feedstock is 1000 TPD (dry basis). The feedstock comprises 11.8% moisture content. The flow of syngas to conditioning reactor 200 comprises 1310 lb/h hydrogen; 40,740 lb/h CO; 23,420 lb/h H₂O; 15,448 lb/h CO₂; 620 lb/h nitrogen; 7,894 lb/h methane; 668 lb/h ethane; 4,606 lb/h ethylene; 46 lb/h ammonia; and 3,032 lb/h naphthalene. The biomass derived syngas has a temperature of 1300° F. and a pressure of 19 psia. Enriched oxygen is fed to syngas conditioning reactor 200 at a flow rate of 20,892 lb/h, a temperature of 400° F., and a pressure of 45 psia. The enriched oxygen comprises 1,852 lb/h N₂ and 19,041 lb/h oxygen. The conditioned synthesis gas outlets reactor 200 at a flow rate of 118,672 lb/h, comprising 4,533 lb/h hydrogen; 65,694 lb/h CO; 21,234 lb/h H₂O; 24,701 lb/h CO₂; 2,509 lb/h nitrogen; 0.27 lb/h methane; and 0.34 lb/h ammonia. The conditioned syngas has a temperature of 2100° F. and pressure of 18 psia. Following compression, the conditioned, compressed syngas has a flow rate of 97,688 lb/h, comprising 4,532.7 lb/h hydrogen; 65,694.1 lb/h CO; 254.8 lb/h H₂O; 24,696.5 lb/h CO₂; 2,509.1 lb/h nitrogen; 0.271 lb/h methane; and 0.26 lb/h ammonia. The conditioned compressed synthesis gas has a temperature of 100° F. and a pressure of 455 psia.

Utility loads are: 2.7 MW for VSA unit, 750 lb/h of 400# saturated steam (for GOX preheater), 9.4 MW for syngas compressor, and 5,400 gpm for cooling water circulation. Steam generation comprises 70,500 lb/h 1000# SH (superheated) steam (exiting HP steam boiler/superheater 202 via line 206) and 11,100 lb/h 75# saturated steam (exiting LP steam boiler 209 via line 210).

The product conditioned synthesis gas has a H₂/CO ratio of 0.96. The product syngas has a concentration of 10.7% CO₂ (dry basis). The product conditioned syngas has a concentration of 12.6% total inerts (including CO₂).

TABLE 1

| | | Syngas | | | |
|---|---|---|---|---|---|
| | | Derived | Enriched | Gas | |
| | | from | Air | Conditioning | Compressor |
| Component | Units | Biomass | from VSA | Outlet | Outlet |
| Hydrogen | lb/h | 1310 | — | 4533 | 4532.7 |
| CO | lb/h | 40740 | — | 65694 | 65694.1 |
| H₂O | lb/h | 23420 | — | 21234 | 254.8 |
| CO₂ | lb/h | 15448 | — | 24701 | 24696.5 |
| Nitrogen | lb/h | 620 | 1852 | 2509 | 2509.1 |
| Methane | lb/h | 7894 | — | 0.27 | 0.271 |
| Ethane | lb/h | 664 | — | — | — |
| Ethylene | lb/h | 4606 | — | — | — |
| Oxygen | lb/h | — | 19041 | — | — |
| Ammonia | lb/h | 46 | — | 0.34 | 0.26 |
| Naphthalene | lb/h | 3032 | — | — | — |
| Total | lb/h | 97780 | 20892 | 118672 | 97688 |
| Temperature | ° F. | 1300 | 400 | 2100 | 100 |
| Pressure | psia | 19 | 45 | 18 | 455 |

Notes:
1. 1000 TPD (dry basis) Feedstock
2. 11.8% Moisture Content in Feedstock
3. 0.96 Product H₂/CO Ratio
4. 10.7% CO₂ Concentration in Product Syngas (dry basis)
5. 12.6% Total Inert Conc. (Including CO₂) in Product Syngas

TABLE 2
Utility Loads

| | |
|---|---|
| VSA Unit, MW | 2.7 |
| Syngas Compressor, MW | 9.4 |
| 400# Sat. Steam, lb/h | 750 |
| Cooling Water Circ., gpm | 5400 |

Steam Generation

| | |
|---|---|
| 1000# SH Steam (700° F.), lb/h | 70500 |
| 75# Sat. Steam, lb/h | 11100 |

While the preferred embodiments of the invention have been shown and described, modifications thereof can be made by one skilled in the art without departing from the spirit and teachings of the invention. The embodiments described herein are exemplary only and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The examples provided in the disclosure are presented for illustration and explanation purposes only and are not intended to limit the claims or embodiment of this invention. While the preferred embodiments of the invention have been shown and described, modifications thereof can be made by one skilled in the art without departing from the spirit and teachings of the invention. Process criteria, equipment, and the like for any given implementation of the invention will be readily ascertainable to one of skill in the art based upon the disclosure herein. The embodiments described herein are exemplary only and are not intended to be limiting. Many variations and modifications of the invention disclosed herein are possible and are within the scope of the invention. Use of the term “optionally” with respect to any element of the invention is intended to mean that the subject element is required, or alternatively, is not required. Both alternatives are intended to be within the scope of the invention.

The discussion of a reference in the Background is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

Although various embodiments of the invention are described herein, it is nevertheless not intended to be limited to the details described, since various modifications and structural changes may be made therein without departing from the spirit of the invention and within the scope and range of equivalents of the claims.

What is claimed is:

1. A thermal conversion process comprising:
providing a first synthesis gas having a first H₂:CO ratio of less than a minimum value or greater than a maximum value;
providing enriched oxygen; and
subjecting the first synthesis gas to partial oxidation in the presence of at least a portion of the enriched oxygen to produce a conditioned synthesis gas having a desired ratio of H₂:CO in the range of from the minimum value to the maximum value.

2. The process of claim 1 wherein the minimum value is about 0.7 and the maximum value is about 2.0.

3. The process of claim 1 wherein the minimum value is about 0.7 and the maximum value is about 1.5.

4. The process of claim 1 wherein the minimum value is about 0.75 and the maximum value is about 1.1.

5. The process of claim 1 wherein the partial oxidation reaction is carried out in a reactor.

6. The process of claim 1 wherein the thermal conversion process is a non-catalytic, high temperature process.

7. The process of claim 6 wherein the high temperature is a temperature in the range of from about 950° C. to about 1400° C.

8. The process of claim 1 further comprising adjusting the portion of enriched oxygen based on a desired H₂:CO ratio.

9. The process of claim 1 wherein providing enriched oxygen comprises providing enriched oxygen at a flow rate in the range of from about 10 lb/h per ton of dry biomass feed to about 100 lb/h per ton of dry biomass feed.

10. The process of claim 1 wherein providing enriched oxygen comprises Vacuum Swing Adsorption (VSA).

11. The process of claim 1 wherein the enriched oxygen comprises from about 50 vol % to about 95 vol % oxygen.

12. The process of claim 11 wherein the enriched oxygen further comprises nitrogen and trace gases present in air.

13. The process of claim 1 wherein providing the first synthesis gas comprises pyrolizing or gasifying a carbonaceous feedstock.

14. The process of claim 13 wherein the first synthesis gas is obtained via gasification.

15. The method of claim 14 further comprising adjusting the moisture content of the first synthesis gas by adjusting the moisture content of the carbonaceous feedstock.

16. The process of claim 13 wherein the carbonaceous feedstock comprises biomass.

17. The process of claim 1 wherein the conditioned synthesis gas is suitable for FT liquids production.

18. The process of claim 17 wherein the conditioned synthesis gas has a H₂:CO ratio on the range of from about 0.75 to about 1.1.

19. The process of claim 17 wherein the conditioned synthesis gas has a H₂:CO ratio on the range of from about 1.5 to about 2.0.

20. The process of claim 1 wherein the first synthesis gas has a H₂:CO ratio in the range of from 0.3 to 1.0 on a dry basis.

21. A method of producing FT product liquids, the method comprising:
(a) providing a conditioned synthesis gas according to the process of claim 1; and
(b) producing FT product liquids by subjecting the conditioned synthesis gas to FT reaction under FT operating conditions.

22. The method of claim 21 further comprising cooling the conditioned synthesis gas via production of high pressure steam, low pressure steam, or a combination thereof.

23. The method of claim 22 further comprising compressing the cooled conditioned synthesis gas prior to (b).

24. The method of claim 21 wherein the tar content in the conditioned synthesis gas is less than 10% of the tar content in the first syngas.

25. A system for conditioning synthesis gas for production of liquid hydrocarbons via FT synthesis, the system comprising:
enriched oxygen production apparatus configured to provide enriched oxygen from air; and
a synthesis gas conditioning reactor fluidly coupled with the enriched oxygen production apparatus, wherein the synthesis gas conditioning reactor is configured for subjecting a first synthesis gas having a first H₂:CO ratio outside a desired range to partial oxidation in the presence of at least a portion of the enriched oxygen to produce a conditioned synthesis gas having a second H₂:CO within the desired range.

26. The system of claim 25 wherein the desired range is from about 0.75 to about 2.

27. The system of claim 25 wherein the enriched oxygen production apparatus comprises vacuum swing adsorption.

28. The system of claim 25 wherein the enriched oxygen comprises from about 50 vol % to about 100 vol % oxygen.

29. The system of claim 25 wherein the synthesis gas conditioning reactor is operable in the absence of catalyst.

30. The system of claim 25 wherein the synthesis gas conditioning reactor is operable at a temperature in the range of from about 950° C. to about 1500° C.

31. The system of claim 25 further comprising synthesis gas production apparatus configured for the production of the first synthesis gas from a carbonaceous material.

32. The system of claim 31 wherein the synthesis gas production apparatus comprises a gasifier.

33. The system of claim 31 wherein the carbonaceous material comprises biomass.

34. The system of claim 25 further comprising at least one FT reactor downstream of the synthesis gas conditioning reactor and configured for the production of FT hydrocarbons from the conditioned synthesis gas.

35. The system of claim 34 wherein the at least one FT reactor comprises FT catalyst.

36. The system of claim 35 wherein the FT catalyst is iron-based.

37. The system of claim 25 further comprising at least one heat exchange device configured for the production of high pressure steam or low pressure steam via heat transfer from the conditioned synthesis gas.
